(19) United States
(12) Patent Application Publication
(10) Pub. No.: US 2018/0318110 A1
(43) Pub. Date: Nov. 8, 2018

(54) TRIAL ACETABULAR LINERS

(71) Applicant: DEPUY IRELAND UNLIMITED COMPANY, COUNTY CORK (IE)

(72) Inventors: SARAH BUSHELL, SHEFFIELD (GB); AHMAD JALANDARI, LEEDS (GB); PETER LANDERS, RAYNHAM, MA (US); RAYMOND LAWTON, FAIRHAVEN, MA (US); CONOR LOWRY, LEEDS (GB); DUNCAN TEMPLE, LEEDS (GB)

(21) Appl. No.: 15/589,088

(22) Filed: May 8, 2017

(51) Int. Cl. A61F 2/46; A61F 2/34; A61B 17/56

(52) U.S. Cl. CPC A61F 2/4684; A61F 2002/30471; A61B 17/56; A61F 2/34

(57) ABSTRACT

A trial liner for fitting in a shell part of an orthopaedic joint prosthesis has a concave inner bearing surface for articulation with a head part, and an opposite outer surface. The liner has first and second portions which are separated by a slot extending between the inner and outer surfaces which has first and second ends. The slot divides the liner into first and second portions which are connected to one another by means of a first hinge at the first end of the slot and a second hinge at the second end of the slot. Each of the first and second portions has a locking feature in the form of a recess or protrusion which can engage a corresponding locking feature on the internal surface of a shell part to lock the liner into the shell part. The hinges allow the first portion of the liner to pivot relative to the second portion between first and second configurations, in which the distance between the first and second portions is less in the second configuration than in the first configuration.

### FIELD OF THE INVENTION

The present invention relates to trial liners for use with orthopaedic implant cup components, kits including the liners and methods of use thereof. In particular, the invention relates to trial acetabular liner for use within the shell part of an acetabular cup component of a hip joint prosthesis.

### BACKGROUND TO THE INVENTION

Joint arthroplasty is often used to restore function to a diseased or injured joint. Examples of joints which can be treated using such techniques include hip and shoulder joints. In total hip arthroplasty, replacement surfaces for the femur and pelvis provided by implant components. In a typical case, the implants include a femoral component having stem and head parts, and an acetabular component. In total shoulder arthroplasty, surfaces of the humerus and the glenoid are prepared and replacement surfaces for the humerus and the glenoid are prepared by implant components. In an anatomic shoulder procedure, a humeral implant component has a convex bearing surface and a glenoid component has a shallow concave bearing surface. In a reverse shoulder procedure, a humeral implant component has a concave bearing surface and a glenoid component has a convex bearing surface.

An acetabular cup component of a hip joint prosthesis typically includes a shell part having substantially hemispherical outer and inner surfaces. The outer surface is generally configured for bone ingrowth to anchor the shell within the acetabulum, and the inner surface receives a liner formed of a bearing material such as polyethylene. The inner surface of the liner functions as a bearing surface for the head part of a femoral component.

During a hip arthroplasty procedure it is often desired to use provisional or trial liners on a trial basis in an assessment of the kinematics and/or range of motion of the joint prior to final fitting of the a liner into a shell. In this procedure, a provisional or trial liner is temporarily secured within the shell. A femoral head part is inserted into the liner. A trial reduction of the joint is then performed. The trial liner may be removed and replaced with other trial liners as necessary until the proper size and/or orientation of the liner is determined. The trial liner is then removed and replaced with a prosthetic liner corresponding to the selected trial liner.

It is known to secure a trial liner temporarily to a shell part of the cup component using a screw, especially a captive screw, which extends through a hole in the liner into a threaded bore in the shell part. This requires use of an instrument to secure the liner in the shell part.

There is a need for a trial liner which is quickly and easily removable from a trial shell or a final/definitive shell, thereby encouraging a surgeon to trial various configurations of the liner of an cup component assembly during surgery in order to optimise the configuration of the assembly.

### SUMMARY OF THE INVENTION

The invention provides a trial liner that can be fitted into a shell part of a cup of an orthopaedic implant cup component, which includes movable hinged portions which enable the trial liner to be easily engaged and disengaged from a shell part.

The invention provides trial acetabular liners for use in combination with a trial shell part or a final/definitive shell part of a cup component during hip arthroplasty. The invention also provides trial liners for use in combination with a trial shell part or a final/definitive shell part used within shoulder arthroplasty. For example, trial humeral liners are received within a humeral implant in reverse shoulder arthroplasty, and trial glenoid liners can be received within a glenoid implant in anatomic shoulder arthroplasty.

The invention provides a trial liner for fitting in a shell part of an orthopaedic joint prosthesis, the liner having a concave inner bearing surface for articulation with a head part, and an opposite outer surface, and having an open face,

the liner having first and second portions which are separated by a slot extending between the inner and outer surfaces which has first and second ends, the slot dividing the liner into first and second portions which are connected to one another by means of a first hinge at the first end of the slot and a second hinge at the second end of the slot, each of the first and second portions having a locking feature in the form of a recess or protrusion which can engage a corresponding locking feature on the internal surface of a shell part to lock the liner into the shell part, the hinges allowing the first portion to pivot relative to the second portion between first and second configurations, in which the distance between the first and second portions measured across the slot is less in the second configuration than in the first configuration.

The reduction of the distance between the first and second portions of the liner when it is in its second configuration allows the locking features on the liner to be disengaged from the corresponding locking features on the shell part so that the liner can be separated from the shell part. The reduction in the distance involves closing the slot at least partially along at least part of its length. For example, when the slot has parallel side walls while the liner is in a relaxed undeformed configuration, the deformation of the liner involves closing the slot at least partially at its mid-point.

Optionally, the locking feature on each of the first and second portions comprises at least one protrusion.

Optionally, the slot is T-shaped at each of the first and second ends. This can increase the flexibility of a living hinge that is provided by the material of the liner adjacent to an end of the slot.

Optionally, the liner has a generally annular collar portion adjacent to the open face of the liner.

Optionally, the slot is provided in the annular collar portion on one side of the liner and in the annular collar portion on the opposite other side of the liner, and in which the first portion of the liner includes a first part of the annular collar portion on one side of the slot and the second portion of the liner includes a second part of the annular collar portion on the other side of the slot.

Optionally, the slot has a first limb which divides the first portion of the liner from the second portion of the liner, a first transverse limb which is formed in the annular collar portion at the first end of the slot and a second transverse limb which is formed in the annular collar portion at the second end of the slot.

Optionally, there is a break in the slot between its first and second ends. The slot can then have first and second parts on first and second sides of the liner. The liner can have at least one opening formed in it which the slot intersects. The first part of the slot can intersect the opening on one side of the opening and the second part of the slot can intersect the opening on an opposite side of the opening.

The slot is defined by first and second opposed edges. Optionally, the liner can have a feature formed in its wall adjacent to each of the opposed edges which allow the liner to be gripped so that a force can be applied to the wall of the liner on opposite sides of the slot to force the edges which define the slot towards one another. It will often be preferred that the feature is a negative feature in the form of a recess or opening. This has the advantage that the feature does not extend into the cavity defined by the liner in which a mating articulation component is received when the liner is in use.

Optionally, the first edge has a protrusion formed on it which extends towards the second edge, and the second edge has a recess formed in it in which the protrusion on the first edge can be received when the edges of the slot are forced towards one another. The protrusion and the recess can be formed in facing edges of first and second arms which extend from the annular collar portion on one side of the liner to the annular collar portion on the opposite other side of the liner. The provision of a protrusion and corresponding recess on the edges which define the slot can provide a visual indication to a user of the possibility of squeezing the edges together to change the configuration of the liner for the purpose of engaging the liner with a shell part or disengaging the liner from a shell part.

The protrusion can be shaped so that it the protrusion is a snug fit in the recess. The shape of the recess will effectively be a negative of the shape of the protrusion. The corresponding shapes of the protrusion and the recess can help a user to recognise that the edges of the slot are to be squeezed together, so that the protrusion is received within the recess.

The protrusion and the recess can be shaped with approximately straight sides. The side edges of the protrusion which slide relative to corresponding edges of the recess can be parallel or taper inwardly slightly (for example with an included angle of not more than about 10°) towards the free end of the protrusion. This can help to control the direction in which the edges of the slot are deformed towards one another.

Optionally, the slot is defined by first and second arms which extend from the annular collar portion on one side of the liner to the annular collar portion on the opposite other side of the liner. The liner can then be changed from the first configuration to the second configuration by applying a force to the first arm relative to the second arm in a direction towards the second arm. The liner can have an opening formed in it on the side of each of the arms which is opposite to the side of that arm which faces the other arm. This facilitates gripping of the arms by a user to apply a force to the arms to squeeze them together.

Optionally, the locking features are formed in the wall of the annular collar portion of the liner.

Optionally, the trial liner includes a spigot which can be received in a bore in a shell part of an orthopaedic joint prosthesis, and in which the slot intersects the spigot so that the spigot is divided into first and second parts so that the first portion of the liner includes a first part of the spigot and the second portion of the liner includes the second part of the spigot.

Optionally, the locking features on the first and second portions of the liner are provided on the first and second parts of the spigot respectively.

The invention also provides a joint prosthesis assembly which comprises a shell part of an orthopaedic joint prosthesis, and a trial liner as discussed above.

The hinges can be living hinges which are provided as one piece with the first and second portions of the liner. The quantity of the material of the liner is smaller in the region of the hinges. The reduced material regions of the liner define the hinges.

Optionally, the movement of the first portion of the liner towards the second portion is limited by contact between surfaces of the first and second portions of the liner on opposite sides of the through slot.

Optionally, the liner can be made from a polymeric material. The inner bearing surface of the liner can be made smooth so that a head part in contact with the inner surface can articulate with the liner. The material for the liner (especially when it is a polymeric material) can be selected for its flexibility so that the hinges can be provided by reduced material regions of the liner. It can be preferred for some applications that the material of the liner is capable of being deformed approximately elastically to a limit on its deformation. A limit on the deformation of the liner might be provided by contact between surfaces of the first and second portions of the liner on opposite sides of the slot.

Examples of suitable polymeric materials for use in the liner include polyolefins such as polyethylenes and polypropylenes and copolymers thereof, polyamides, polyesters, and polycarbonates.

The invention also provides a trial liner for use in an orthopaedic implant cup component, the liner being removably receivable within the shell part of cup component; the liner comprising an inner bearing surface being generally concave and forming a cavity adapted to receive a generally convex head component and an outer surface adapted to engage with an inner surface of the shell part, the outer surface including a locking mechanism for reversibly locking the liner to the shell part, the liner further including a through slot within the inner bearing surface dividing the liner into a movable segment comprising at least part of the inner bearing surface, in which the movable segment can be displaced about a hinge portion thereby forcing the locking mechanism into locking engagement with the shell part.

Optionally, the liner is substantially annular and includes a proximal end surface and a distal end surface, a first hinge portion located at the distal end surface from which a first through slot extends into the proximal end surface, and a second hinge portion located at the distal end surface from which a second through slot extends into the proximal end surface, thereby forming first and second movable segments. In some constructions, the first hinge portion is located on a first side of the liner, and the second hinge portion is located on a second side of the liner, the second side being opposite to the first side.

In order to increase the ease with which the trial liner can be inserted and removed from a shell part of a cup component of a joint prosthesis, each of the first and second movable segments may include a graspable member configured to be grasped by a user so as to displace each movable segment. Each graspable member may be in the form of a leg that extends from the proximal end surface of the liner and extends at least partly between the first side and the second side of the liner. Optionally, the leg may have a substantially concave inner surface. This prevents the graspable member from disrupting the seating of a head part of an articulating component within the liner. In some constructions, each graspable member extends from the first side of the liner to join the second side of the liner. The graspable members may be pinched to together by a user, for example between the thumb and finger, or using a surgical instrument, such as forceps.

The through slot may be T-shaped. The so-called “live spring” action of the trial liner can also be tailored through the use of different shapes and placement of the through slot. A consideration when choosing the shape and placement of the through slot is that the material should be prevented from reaching its yield point. The yield point is the material property defined as the stress at which material begins to deform plastically. Prior to the yield point the material will deform elastically and will return to its original shape when an applied stress is removed. Once the yield point is passed, some fraction of the deformation will be permanent and non-reversible.

Advantageously, the geometry of the through slot should also provide uniform resistance to a pinch force across a range of trial liners. This provides the user with the same surgical experience when trialling with different liners.

Optionally, the locking mechanism comprises at least one positive surface feature formed on the outer surface of the liner that is reversibly mateable with a corresponding negative surface feature formed in the inner surface of the cup.

A non-limiting example of a suitable positive surface feature is a barb. In order to ensure a secure engagement between the liner and the cup, a plurality of barbs may be distributed about the outer surface of the liner. The or at least one of the plurality of barbs are configured to grip reversibly within the corresponding negative surface feature.

A non-limiting example of a suitable corresponding negative surface feature is a groove. In some constructions, the inner surface of the cup includes a circumferential groove.

In some constructions, the positive surface feature on the outer surface of the liner includes a series of barbs and the corresponding negative feature on the inner surface of the cup is a circumferential groove. The barbs may be substantially triangular. Advantageously, the distal face of the barb is generally planar relative to the outer surface of the liner. This improves the retention of the barb within the groove and reduces the risk that the liner will be pulled out in an axial direction. The barb also has a lead-in face, which is preferably angled. This contributes to the folding motion of the liner, and is of particular use if the user has not sufficiently pinched the graspable members (e.g., legs) together.

Optionally, the locking mechanism comprises at least one negative surface feature formed on the outer surface of the liner which can be mated reversibly with a corresponding positive surface feature formed in the inner surface of the cup.

A non-limiting example of a suitable negative surface feature is a groove. In order to ensure a secure engagement between the liner and the cup, a groove may be distributed about the outer surface of the liner. The groove may be in the form of a circumferential groove extending part or fully about the outer surface of the liner. Optionally, a plurality of grooves may be circumferentially distributed around the outer surface of the liner. The or at least one of the plurality of grooves is/are configured to reversibly mate with a corresponding positive surface feature on the inner surface of the cup.

A non-limiting example of a suitable corresponding positive surface feature is a barb. In some constructions, the inner surface of the cup can include a plurality of barbs.

In some constructions, the positive surface feature on the inner surface of the cup is a series of barbs and the corresponding negative feature on the outer surface of the cup is a circumferential groove. The barbs may be substantially triangular.

In some other constructions of the trial liner, the liner is generally hemispherical and includes a polar region having a pole and first and second through slots extending across a part of the polar region, the slots defining a hinge portion between them about which a movable segment can be displaced.

Optionally, the slots define a second hinge portion between them about which a second movable segment can be displaced.

Optionally, each of the first and second through slots is T-shaped. It is envisaged that other shaped through slots may be used, and that the skilled person would readily identify suitable slot shapes that would result in the first and second movable segments being capable of being displaced outwardly about a hinge portion.

The locking mechanism may comprise a projection extending outwardly from the pole of the liner.

In order to reduce or prevent relative rotation of the liner relative to a shell part, the outer surface of the liner can include an anti-rotation element. Acetabular liners typically include a series of anti-rotation element or anti-rotation devices (ARDs) in the form of radial projections (e.g., ARD tabs) disposed about the outer surface which are receivable within a corresponding series of spaced recesses (e.g., ARD scallops) disposed on a shell part when the liner is seated within the shell part.

The invention also provides a kit comprising a plurality of trial liners. The liners can have any of the features which are discussed above. Liners in the kit can differ from one another in terms of size. Liners in the kit can differ from one another in terms of other shape features such as offset. Each of the liners in the kit can be fitted into a common shell part (which can be a trial shell part or a final or definitive implant shell part).

The trial liners can be liner parts of a shell part of an acetabular component of a hip joint, which are configured to receive the head part of a femoral component.

The invention also provides a method of trialling a cup component of an orthopaedic joint prosthesis which includes a shell part and a liner, the method comprising:
using a trial liner comprising an inner bearing surface being generally concave and defining a cavity adapted to receive a generally convex head component and an outer surface adapted to engage with an inner surface of the shell part, the outer surface including a locking mechanism for reversibly locking the liner to the shell part, the liner further including a through slot within the inner bearing surface dividing the liner into a movable segment, in which the movable segment can be displaced about a hinge portion, thereby forcing the locking mechanism into locking engagement with the shell part,
inserting the trial liner into a shell part of the cup component, the movable segment moving during the insertion in a first direction such that the locking mechanism engages with the shell part,
inserting a head part into the inner cavity of the liner,
performing a trial reduction,
conducting a trial range of motion,
extracting the head part, and
moving the movable segment in a second direction which is opposite to the first direction such that the locking mechanism becomes disengaged from the cup, and
extracting the trial liner from the shell part.

The trial liner may be inserted into a trial shell part or into a final implant shell part.

Optionally, the method shell part is a shell part of an acetabular cup component of a hip joint prosthesis and the head part is the head part of a femoral component of a hip joint prosthesis.

In some examples of the trialling method, the trial liner is substantially annular with a proximal end surface and a distal end surface, and a first hinge portion located at the distal end surface from which a first through slot extends into the proximal end surface, and a second hinge portion located at the distal end surface from which a second through slot extends into the proximal end surface, in which each of the first and second movable segments includes a graspable member, and in which the step of inserting and/or extracting the trial liner into/from the shell part (for example the shell part of an acetabular cup component) comprises a user grasping the graspable member and applying a pinching motion to displace the movable segment in the second direction. The user may apply the pinching motion using their digits or a surgical tool, such as forceps.

### DETAILED DESCRIPTION OF THE INVENTION

The invention will now be described, by way of example only, with reference to the following drawings in which:

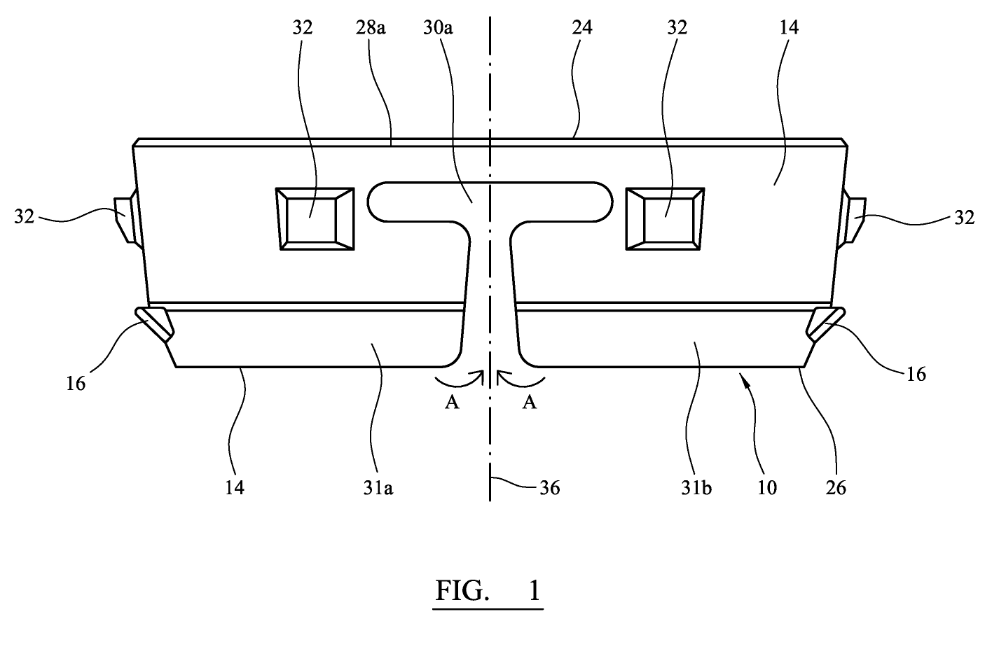

FIG. 1 is a side elevation view of a first construction of the trial acetabular liner.

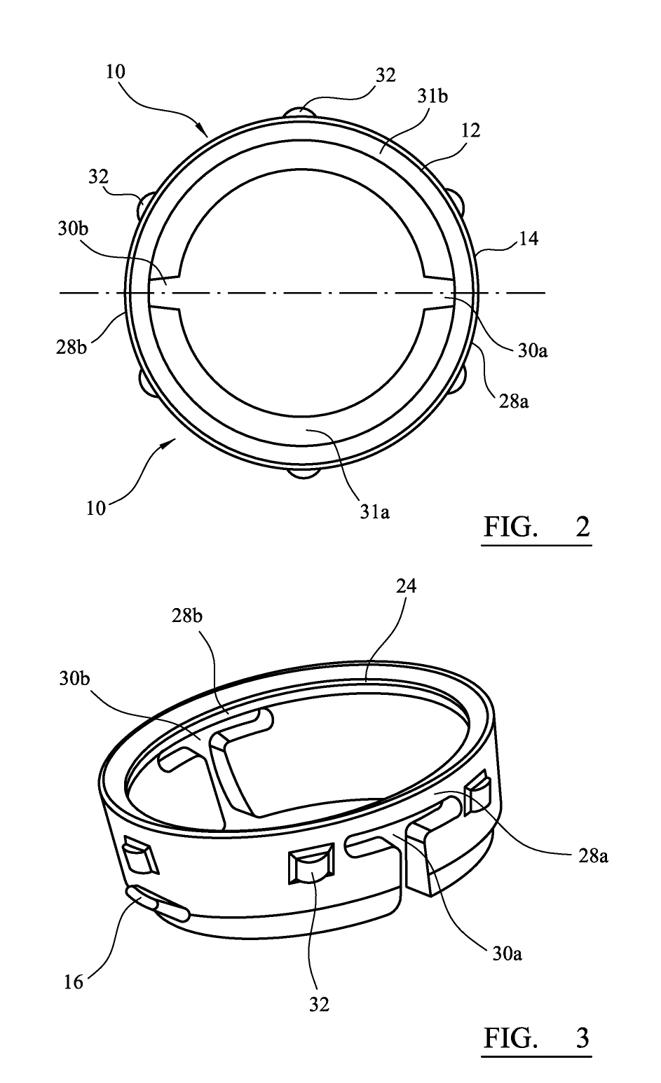

FIG. 2 is bottom elevation view of the liner of FIG. 1.

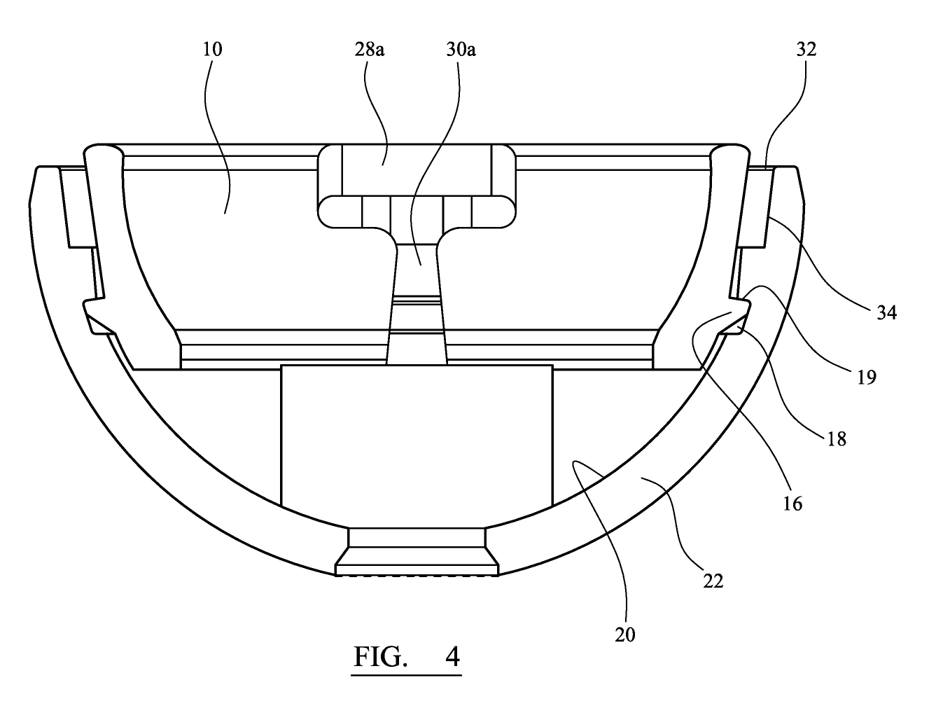

FIG. 3 is an isometric view of the liner of FIG. 1.

FIG. 4 is a schematic showing the liner of FIG. 1 seated within the shell part of an acetabular cup.

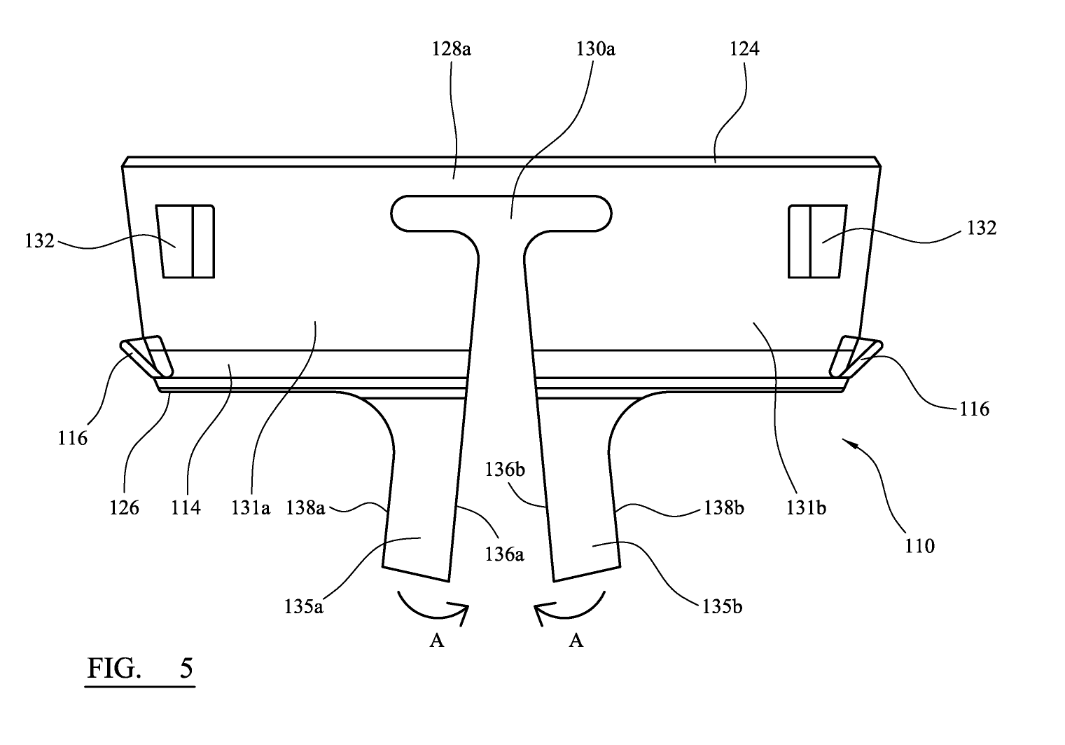

FIG. 5 is a side elevation view of a second construction of the trial acetabular liner.

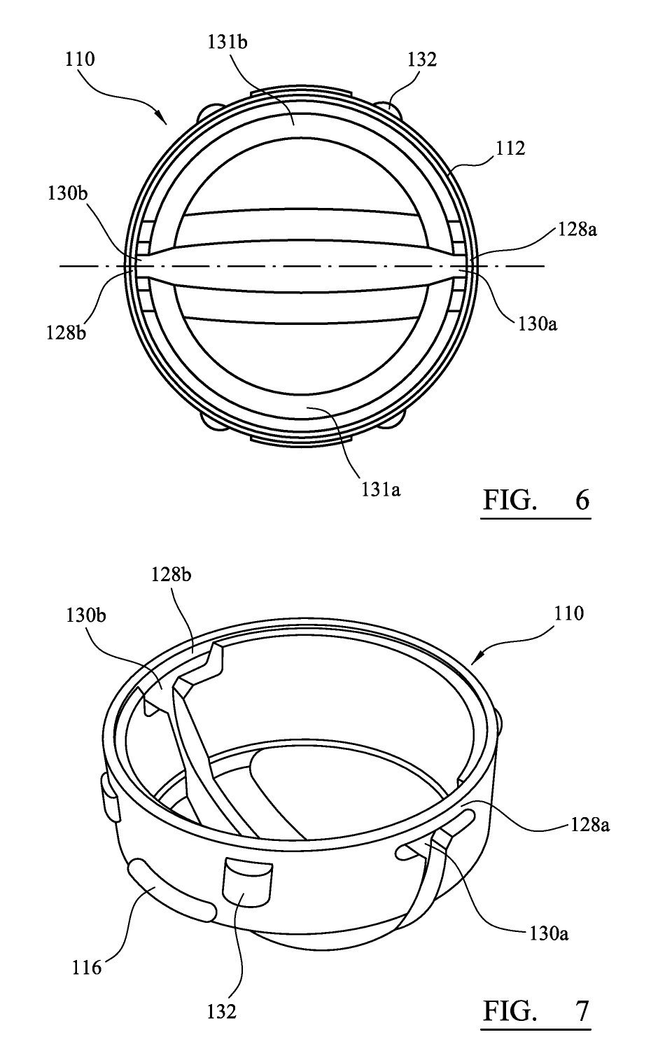

FIG. 6 is bottom elevation view of the liner of FIG. 5.

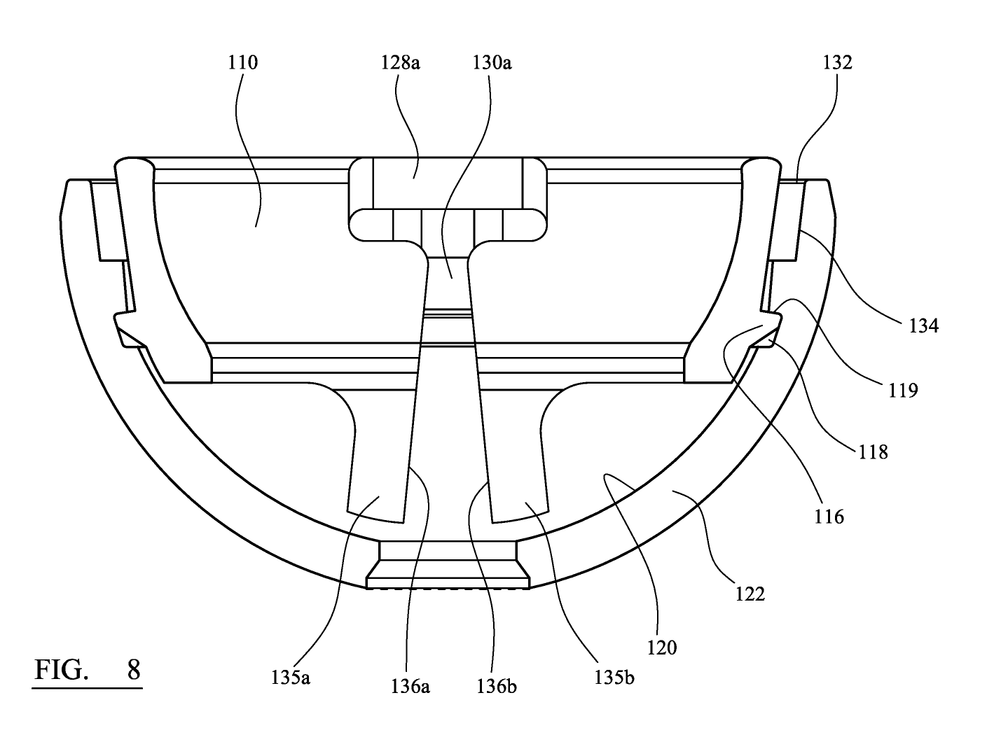

FIG. 7 is an isometric view of the liner of FIG. 5.

FIG. 8 is a schematic showing the liner of FIG. 5 seated within the shell part of an acetabular cup.

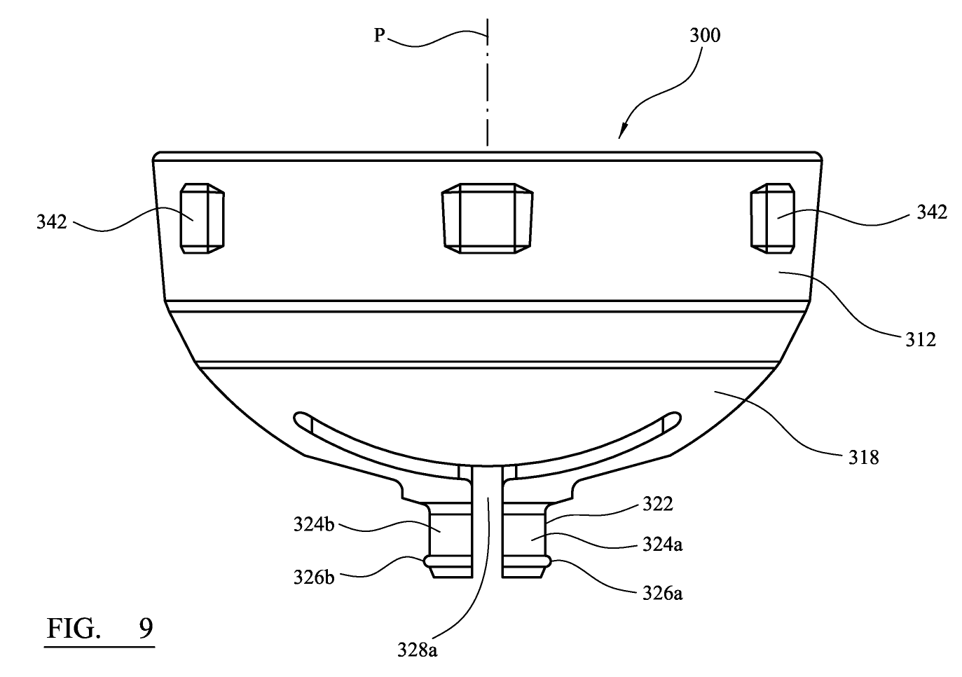

FIG. 9 is a side elevation view of a third construction of the trial acetabular liner.

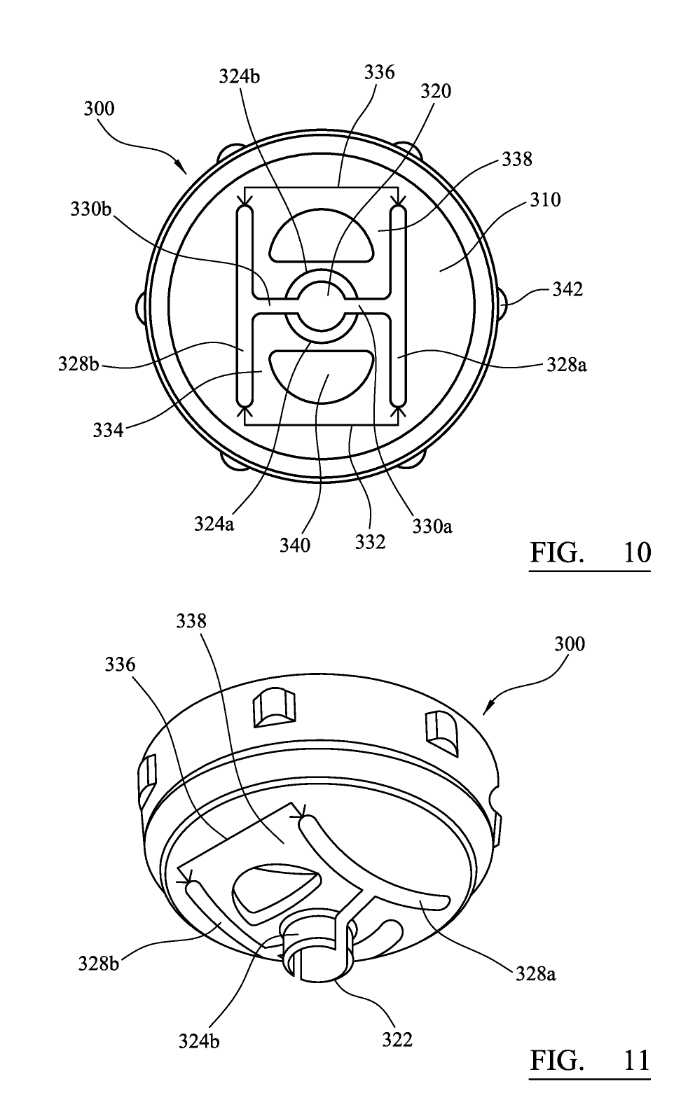

FIG. 10 is bottom elevation view of the liner of FIG. 9.

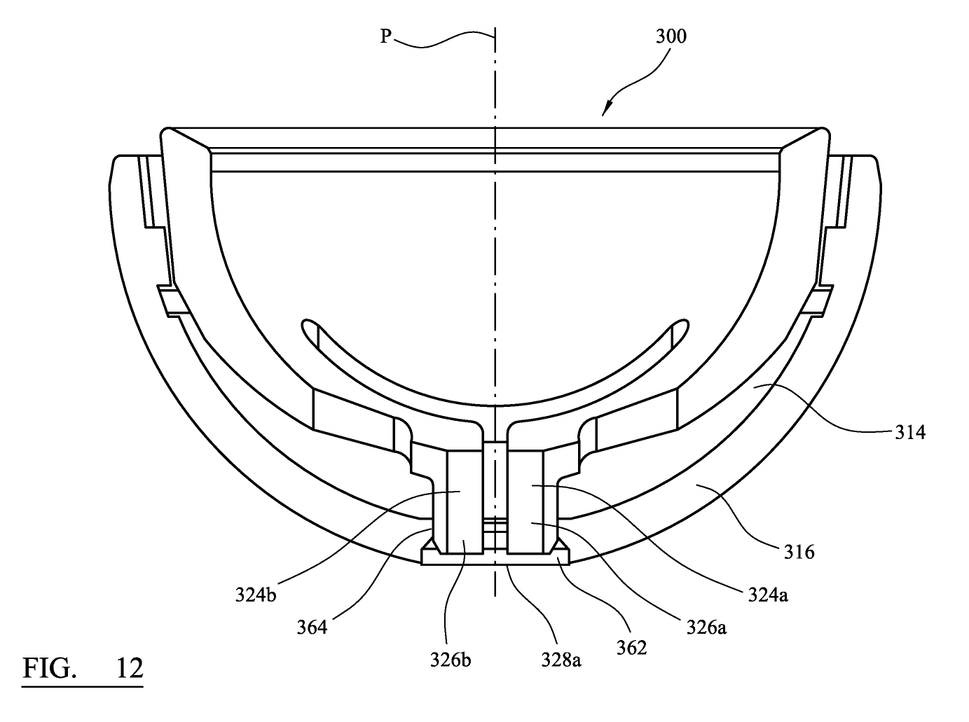

FIG. 11 is an isometric view of the liner of FIG. 9.

FIG. 12 is a schematic showing the liner of FIG. 9 seated within the shell part of an acetabular cup.

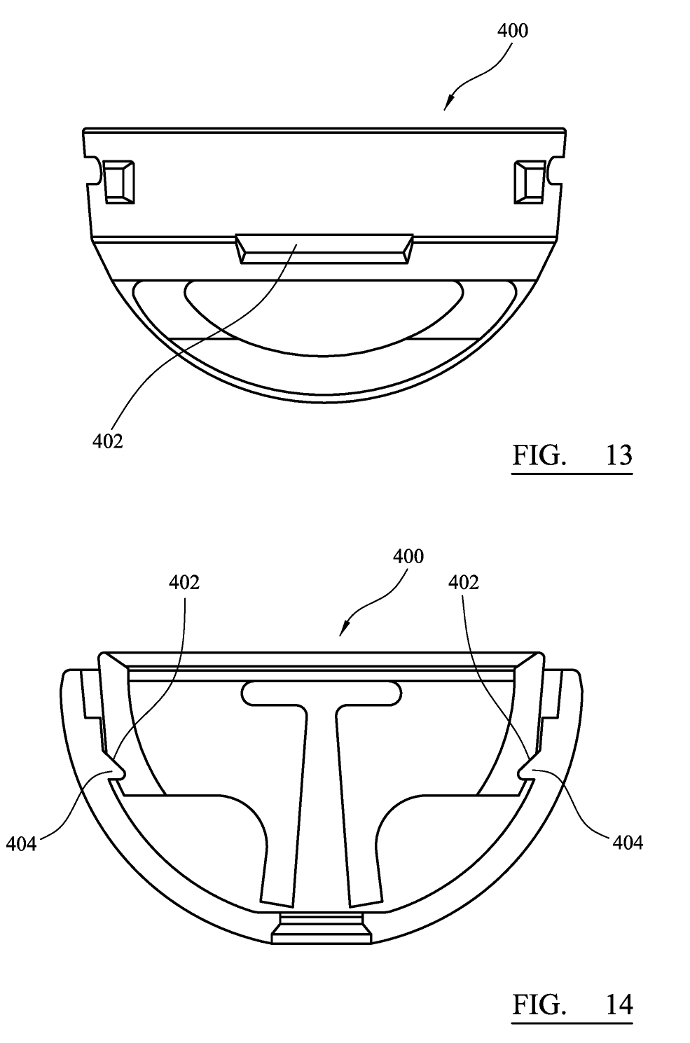

FIG. 13 is a side elevation view of a fourth construction of the trial acetabular liner.

FIG. 14 is a schematic showing the liner of FIG. 13 seated within the shell part of an acetabular cup.

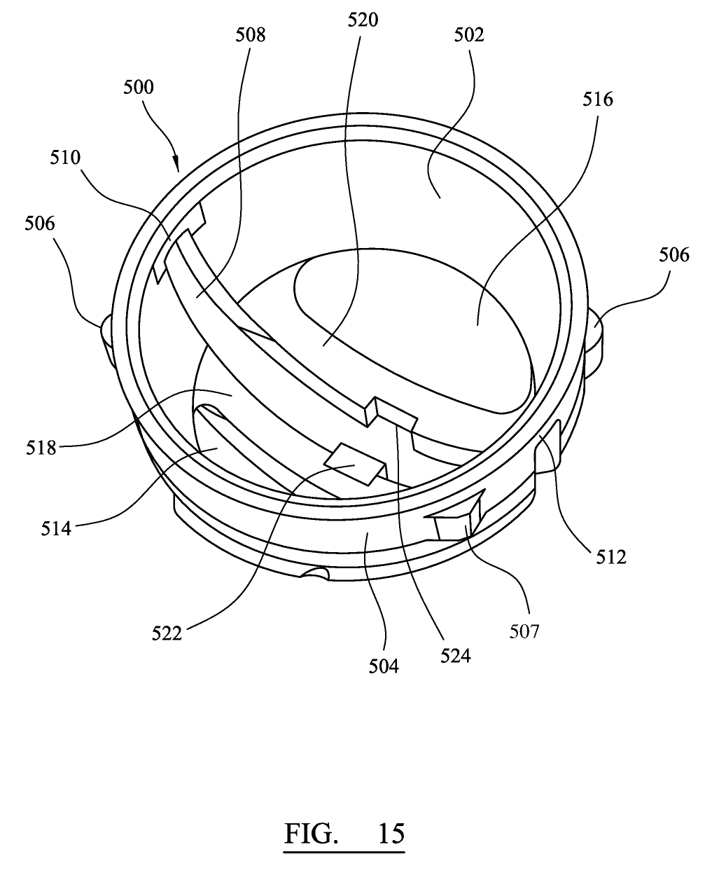

FIG. 15 is an isometric view of a trial acetabular liner in which the arms which define the slot have mating protrusion and recess features.

Referring to the drawings, FIGS. 1 to 4 show a trial acetabular liner 10. The liner 10 is substantially annular and includes an inner bearing surface 12 and an outer surface 14. The inner surface 12 is generally concave and forms a cavity adapted to receive a femoral head. The outer surface 14 is provided with a plurality of barbs 16 extending outwardly. The barbs are configured to engage with a circumferential groove 18 provided on an inner surface 20 of the shell part 22 of an acetabular cup component. As shown in FIG. 4, the groove 18 has an undercut 19. Advantageously each barb is triangular, with a distal face that is generally planar. The distal flat face engages with the undercut 19 of the groove, thereby limiting axial movement of the liner.

The liner also includes a distal end surface 24 and a proximal end surface 26. A first hinge portion 28a is located at the distal end surface 24 on a first side of the liner. A first T-shaped through slot 30a extends proximally from the hinge portion 28a into the proximal end surface 26. A second hinge portion 28b is located at the distal end surface 24 on a second side of the liner. A T-shaped through slot 30b extends proximally from the hinge portion 28 into the proximal end surface. The first and second through slots are located directly opposite each other. Slots 30a and 30b divide the liner into a first movable segment 31a and a second movable segment 31b.

A series of anti-rotation devices (ARDs) in the form of tabs 32 project from the outer surface 14. The tabs 32 are located distally of the barbs 16. As shown in this construction, the tabs may be generally semi-circular. The tabs are received within a series of spaced recesses 34 disposed on the acetabular cup 22. This prevents relative rotation of the liner and the cup.

The liner is provided to the user in a so-called “relaxed” state. The stiffness of the material of at least the hinge portions 28a, 28b of the liner is selected such that the first and second movable segments 31a, 31b can flex resiliently about the respective hinge portions 28a, 28b when a force is applied in the direction of arrows A to move the first segment and/or the second segment toward the cup axis 36 (for example by the application of a pinching motion by a user). The segments 31a, 31b are configured to flex inwardly about the hinge portions. The deformation characteristics of the material of the liner mean that the first and second movable segments 31a, 31b return substantially to their natural “relaxed” configuration when the deforming force is removed.

A user inserts the liner into the acetabular cup by moving the first and second movable segments 31a, 31b inwardly towards one another (in the direction of arrows A). The liner is then inserted into the shell part of the acetabular cup with the barbs in line with the circumferential groove 18 and the tabs 32 in line with corresponding recesses 34. As the user releases tension on the liner, the first and second movable segments 31a, 31b move outwardly, to return to the “relaxed” state. This results in the barbs being received in the groove 18.

A head part of a femoral component located within the cavity defined by the inner bearing surface 12 prevents inward deformation of the first and second segments 31a, 31b towards one another. This keeps the barbs in engagement with the groove 18 and prevents the disassembly of the liner from the shell part.

To extract the liner from the shell part, the user again moves the first and second movable segments 31a, 31b inwardly (in the direction of the arrows A). This releases the barbs from the groove. The liner can then be extracted.

FIGS. 5 to 8 show another construction of the liner 110. The liner 110 is similar to the embodiment shown and described with reference to FIGS. 1 to 4 with like parts bearing similar reference numerals indexed by 100. The liner 110 is substantially annular and includes an inner bearing surface 112 and an outer surface 114. The inner surface 112 is generally concave and forms a cavity adapted to receive a femoral head. The outer surface 114 is provided with a plurality of outwardly extending barbs 116. The barbs are configured to engage with a circumferential groove 118 provided on an inner surface 120 of the shell part 122 of an acetabular cup. As shown in FIG. 4, the groove has an undercut 119. Advantageously each barb is triangular, with a distal face that is generally planar. The distal flat face engages with the undercut 119 of the groove, thereby limiting axial movement of the liner.

The liner also includes a distal end surface 124 and a proximal end surface 126. A first hinge portion 128a is located at the distal end surface 124 on a first side of the liner. A first part 130a of a slot extends proximally from the hinge portion 128a into the proximal end surface. A second hinge portion 128b is located at the distal end surface 124 on a second side of the liner. A second part 130b of a slot extends proximally from the hinge portion 128b into the proximal end surface. The first part 130a of the slot is located opposite to the second part of the slot. Each part of the slot is T-shaped. The slot (provided by the first and second part 130a, 130b) divides the liner into a first movable segment 131a and a second movable segment 131b.

A series of anti-rotation devices (ARDs) in the form of tabs 132 project from the outer surface 114 of the liner. The tabs 132 are located distally of the barbs 116. As shown in this construction, the tabs may be generally semi-circular. The tabs are received within a series of spaced recesses 134 disposed on the internal surface of the shell part 122. This prevents relative rotation of the liner and the cup.

In order to aid in the flexing of the first and second movable segments 131a, 131b about hinge portions 128a, 128b, respectively, the proximal end surface 126 of each segment is provided with a graspable member 135a, 135b. Each graspable member is configured to be grasped by the user's digits or a surgical tool (e.g., forceps). In the construction shown, each graspable member is in the form of an arcuate-shaped leg. Each leg 135a, 135b extends between the proximal end surface 126 of a first side of the liner to an opposing second side of the liner. Optionally, each leg has a concave inner surface which functions as an articulating/bearing surface for an inserted femoral head. Each leg also includes a medial surface 136a, 136b and a lateral surface 138a, 138b. Pinching of the legs together in the direction of the arrows A moves the medial surfaces 136a, 136b towards each other. This results in the first and second movable segments 131a, 131b flexing inwardly about hinge portions 128a, 128b, respectively. When a user releases the pinching motion, the legs 135a, 135b return substantially to their original positions.

A third embodiment of the trial acetabular liner is shown in FIGS. 9 to 12. The liner 300 is generally hemispherical and includes an inner surface 310 and an outer surface 312. The inner bearing surface 310 is generally concave and forms a cavity adapted to receive a head part of a femoral component of a hip prosthesis. The outer surface 312 is generally convex and is adapted to engage with an inner surface 314 of the shell part 316. The liner includes a polar region 318 having a pole 320 with a pole axis P.

A spigot 322 extends outwardly from the outer surface 312 of the liner along the pole axis P. The spigot shown in this particular construction includes two substantially aligned legs 324a, 324b, separated by a slot. Each leg includes a hook-like proximal end surface 326a, 326b that is configured to be secured within the polar hole of a shell part, as will be described further below.

A first T-shaped part 328a of a slot extends outwardly from a first side 330a of the legs 324a, 324b. A second T-shaped part 328b of the slot extends outwardly from a second side 330b of legs 324a, 324b, opposite to the first side 330a. A first hinge portion 332 is defined between an end of the first T-shaped part 328a of the slot and an end of the second T-shaped part 328b of the slot. This provides a first movable segment 334 that can flex about the first hinge portion 332. Flexing of the first movable segment 334 results in movement of leg 324a relative to the pole axis and thereby into and out of locking engagement with a polar hole in a shell part of an acetabular cup prosthesis. A second hinge portion 336 is defined between an end of the first T-shaped part 328a of the slot and an end of the second T-shaped part 328b of the slot, the ends being opposite ends to the ends defining the first hinge portion. This provides a second movable segment 338 that can flex about the second hinge portion 336. Flexing of the second movable segment 338 results in movement of leg 324b relative to the pole axis and thereby into and out of locking engagement with a polar hole in a shell part of an the acetabular cup prosthesis.

An aperture 340 is located within the polar region of the liner and close to the first and/or second hinge portion. The aperture reduces the amount of material at the hinge portion. This improves the ease with which the first and/or second movable segment can be deflected about the hinge portion. In the construction shown, a substantially D-shaped aperture is provided on each side of the pole between the first and second T-shaped parts of the slot. The apertures can also help the user to move at least one of the first and/or second movable segments. A user may introduce the end of a tool or a finger into the aperture to help move the segment about the hinge portion. The provision of an aperture within each movable segment is particularly advantageous as this helps the user to apply a pinching motion.

A series of anti-rotation devices (ARDs) in the form of tabs 342 project from the outer surface 314. As shown in this construction, the tabs may be generally semi-circular. The tabs are received within a series of spaced recesses (not shown) disposed on the acetabular cup 316. This prevents relative rotation of the liner and the cup.

The liner is provided to the user in a so-called “relaxed” state. The deformation characteristics of the material of at least the hinge portions 324a, 324b of the liner mean that the first and second movable segments 334, 338 can flex about their respective hinge portions when a deforming force is applied (for example by the application of a pinching motion by a user). It is preferred that the deformation characteristics of the material of the liner mean that the first and second movable segments 334, 338 can return at least partially toward their natural “relaxed” state when the deforming force is removed. Preferably, the movable segments return substantially completely to their natural “relaxed” state when the deforming force is removed.

A user inserts the liner 300 into the shell part of an acetabular cup by applying a pinching motion to move the respective legs 324a, 324b on the first and second movable segments 334 and 338 towards the polar axis. This enables the legs to be inserted into the polar hole 362 of a shell part. On removal of the pinching motion, the legs return towards their un-tensioned, “relaxed” state. The hook-like proximal end surfaces 326a, 326b engage with an undercut 364 in the polar hole of the acetabular cup. Once the femoral head has been inserted, the first and second movable segments 334, 338 are unable to be moved. This keeps the hook-like proximal end surfaces 326a, 326b in engagement with the undercut 364 and prevents the disassembly of the liner from the shell part.

To extract the liner from a shell part, the user again moves the respective legs 324a, 324b on the first and second movable segments 334 and 338 towards the polar axis by applying a pinching motion. This disengages the hook-like proximal end surfaces 326a, 326b from the undercut 364. The legs are then able to be extracted from the polar hole of the acetabular cup, allowing the liner to be removed.

A fourth embodiment of the trial acetabular liner 400 is shown in FIGS. 13 and 14. The design of the fourth embodiment is similar to the design of the second embodiment. However, in the fourth embodiment the mating connection between the trial liner and the shell part of an acetabular cup is formed between a plurality of negative features on the outer surface of the liner that are adapted to matingly receive a plurality of positive features on the inner surface of the shell part. As shown, the plurality of negative features include a series of grooves 402 distributed circumferentially about the outer surface of the liner. The grooves are adapted to matingly receive corresponding barbs 404 that are distributed about the inner surface of the liner.

FIG. 15 shows a trial acetabular liner 500 having an inner bearing surface 502 and an outer surface 504. The inner surface is concave and forms a cavity which can receive a femoral head. The outer surface has outwardly projecting tabs 506 which can engage corresponding recesses in the inside wall of a shell part of a cup component of a hip joint prosthesis. A series of anti-rotation devices (ARDs) in the form of tabs 507 project from the outer surface.

A slot 508 is formed in the liner which extends through the slot between its inner and outer surfaces. The slot extends across the pole region of the liner between spaced apart ends which are located close to the equator of the liner. The portions 510, 512 of the wall of the liner between the ends of the slot and the equator act as living hinges.

Openings 514, 516 are provided in the wall of the liner, with one opening on each side of the slot 508, so that the slot is effectively defined by first and second arcuate arms 518, 520 which extend between the hinge portions 510, 512.

The first arm 518 has a protrusion 522 formed in it on the edge which faces towards the second arm 520. The second arm 520 has a matching recess 524 formed in it on the edge which faces towards the first arm 518. The protrusion has side walls which are approximately parallel to one another. The protrusion is a snug fit in the recess when the first and second arms 518, 520 are squeezed towards one another. Squeezing the arms towards one another causes the liner to deform at the hinge portions at the end of the slot, so that the distance between first and second portions of the liner on opposite sides of the slot is reduced. This can facilitate engagement of the liner with a shell part, and also subsequent disengagement from the shell part.

It will be noted that alternative embodiments of each of the systems of the present disclosure may not include all of the features described yet still benefit from at least some of the advantages of such features. Those of ordinary skill in the art may readily devise their own implementations of a system that incorporates one or more of the features of the present disclosure and fall within the spirit and scope of the invention as defined by the appended claims.

1. A trial liner for fitting in a shell part of a cup component of an orthopaedic joint prosthesis, the liner having a concave inner bearing surface for articulation with a head part, and an opposite outer surface, and having an open face,
the liner having first and second portions which are separated by a slot extending between the inner and outer surfaces which has first and second ends, the slot dividing the liner into first and second portions which are connected to one another by means of a first hinge at the first end of the slot and a second hinge at the second end of the slot, each of the first and second portions having a locking feature in the form of a recess or protrusion which can engage a corresponding locking feature on the internal surface of a shell part to lock the liner into the shell part, the hinges allowing the first portion to pivot relative to the second portion between first and second configurations, in which the distance between the first and second portions measured across the slot is less in the second configuration than in the first configuration.

2. A trial liner as claimed in claim 1, in which the locking feature on each of the first and second portions comprises at least one protrusion.

3. A trial liner as claimed in claim 1, in which the slot is T-shaped at each of the first and second ends.

4. A trial liner as claimed in claim 1, in which there is a break in the slot between its first and second ends.

5. A trial liner as claimed in claim 1, in which the slot is defined by first and second opposed edges, and in which the first edge has a protrusion formed on it which extends towards the second edge, and the second edge has a recess formed in it in which the protrusion on the first edge can be received when the edges of the slot are forced towards one another.

6. A trial liner as claimed in claim 1, in which the liner has a generally annular collar portion adjacent to the open face of the liner.

7. A trial liner as claimed in claim 6, in which the slot is provided in the annular collar portion on one side of the liner and in the annular collar portion on the opposite other side of the liner, and in which the first portion of the liner includes a first part of the annular collar portion on one side of the slot and the second portion of the liner includes a second part of the annular collar portion on the other side of the slot.

8. A trial liner as claimed in claim 7, in which the slot has a first limb which divides the first portion of the liner from the second portion of the liner, a first transverse limb which is formed in the annular collar portion at the first end of the slot and a second transverse limb which is formed in the annular collar portion at the second end of the slot.

9. A trial liner as claimed in claim 6, in which the slot is defined by first and second arms which extend from the annular collar portion on one side of the liner to the annular collar portion on the opposite other side of the liner, and in which the liner can be changed from the first configuration to the second configuration by applying a force to the first arm relative to the second arm in a direction towards the second arm.

10. A trial liner as claimed in claim 6, in which the locking features are formed in the annular collar portion of the liner.

11. A trial liner as claimed in claim 1, which includes a spigot which can be received in a bore in a shell part of an orthopaedic joint prosthesis, and in which the slot intersects the spigot so that the spigot is divided into first and second parts so that the first portion of the liner includes a first part of the spigot and the second portion of the liner includes the second part of the spigot.

12. A trial liner as claimed in claim 11, in which the locking features on the first and second portions of the liner are provided on the first and second parts of the spigot respectively.

13. A joint prosthesis assembly which comprises:
a. a shell part of an orthopaedic joint prosthesis, and
b. a trial liner as claimed in claim 1.

14. A trial liner for fitting into a shell part of a cup component of an orthopaedic joint prosthesis, the liner being removably receivable within the shell part; the liner comprising an inner bearing surface being generally concave and forming a cavity adapted to receive generally convex head component and an outer surface adapted to engage with an inner surface of the shell part, the outer surface including a locking mechanism for reversibly locking the liner to the shell part, the liner further including a through slot within the inner bearing surface dividing the liner into a movable segment comprising at least part of the inner bearing surface, in which the movable segment can be displaced about a hinge portion thereby forcing the locking mechanism into locking engagement with the shell part.

15. The trial liner according to claim 14, in which the liner is substantially annular with a proximal end surface and a distal end surface, and in which a first portion of the through slot intersects the proximal end surface and defines a first hinge portion between the first portion of the through slot and the distal end surface, and a second portion of the through slot intersects the proximal end surface opposite to the first portion of the through slot and defines a second hinge portion between the second portion of the through slot and the distal end surface, the first and second portions of the through slot defining first and second movable segments.

16. The trial liner according to claim 15, in which each of the first and second movable segments includes a graspable member configured to be grasped by a user so as to displace each movable segment.

17. The trial liner according to claim 16, in which each graspable member comprises a leg extending from the proximal end surface and extending at least partly between the first side and the second side of the liner, each leg having a substantially concave inner surface.

18. The trial liner according to claim 14, in which the liner is generally hemispherical and includes a polar region having a pole and a through slot extending across the polar region, the slot defining a hinge portion between them about which a movable segment can be displaced.

19. The trial liner according to claim 18, in which the through slot is T-shaped at each end.

20. The trial liner according to claim 19, in which the locking mechanism comprises a spigot extending outwardly from the pole of the liner.

21. A method of trialling a cup component of an orthopaedic joint prosthesis which includes a shell part and a liner, the method comprising:
using a trial liner comprising an inner bearing surface being generally concave and defining a cavity adapted to receive a generally convex head component and an outer surface adapted to engage with an inner surface of the shell part, the outer surface including a locking mechanism for reversibly locking the liner to the shell part, the liner further including a through slot within the inner bearing surface dividing the liner into a movable segment, in which the movable segment can be displaced about a hinge portion, thereby forcing the locking mechanism into locking engagement with the shell part,
inserting the trial liner into a shell part of the cup component, the movable segment moving during the insertion in a first direction such that the locking mechanism engages with the shell part,
inserting a head part into the inner cavity of the liner,
performing a trial reduction,
conducting a trial range of motion,
extracting the head part, and
moving the movable segment in a second direction which is opposite to the first direction such that the locking mechanism becomes disengaged from the shell part, and
extracting the trial liner from the shell part.
